(19) United States
(12) Patent Application Publication
(10) Pub. No.: US 2015/0361400 A1
(43) Pub. Date: Dec. 17, 2015

(54) COMPOSITIONS AND METHODS FOR MAINTAINING AND IMPROVING PANCREATIC ISLET CELL FUNCTION AND STABILITY

(71) Applicant: WAKE FOREST UNIVERSITY HEALTH SCIENCES, Winston-Salem, NC (US)

(72) Inventors: Peter Anthony Antinozzi, Winston-Salem, NC (US); Ellen Nicole Dover, Winston-Salem, NC (US)

(21) Appl. No.: 14/763,302

(22) PCT Filed: Jan. 27, 2014
(86) PCT No.: PCT/US14/13182

§ 371 Date: Jul. 24, 2015

Related U.S. Application Data
(60) Provisional application No. 61/757,071, filed on Jan. 25, 2013.

(51) Int. Cl. C12N 5/071; A61K 35/39

(52) U.S. Cl. CPC C12N 5/0676; A61K 35/39; C12N 2501/999

(57) ABSTRACT

Compositions and methods for production of beta islet cells exhibiting superior endocrine like functions are disclosed.

This application claims priority to U.S. Provisional Application No. 61/757,071 filed Jan. 25, 2013, the entire contents being incorporated herein by reference as though set forth in full.

Pursuant to 35 U.S.C. §202(c) it is acknowledged that the U.S. Government has rights in the invention described herein, which was made in part with funds from the National Institutes of Health, Grant Number DK080151.

### FIELD OF THE INVENTION

This invention relates to the fields of diabetes and islet cell transplantation. More specifically, the invention provides improved compositions and methods having therapeutic efficacy for the treatment of diabetes mellitus.

### BACKGROUND OF THE INVENTION

Several publications and patent documents are cited throughout the specification in order to describe the state of the art to which this invention pertains. Each of these citations is incorporated herein by reference as though set forth in full.

Type I diabetes (T1D) results from the autoimmune destruction of pancreatic beta cells, a process believed to be strongly influenced by multiple genes and environmental factors. The incidence of T1D has been increasing in Western countries and has more than doubled in the United States over the past 30 years. The disease shows a strong familial component, with first-degree relatives of cases being at 15 times greater risk of T1D than a randomly selected member of the general population and monozygotic twins being concordant for T1D at a frequency of approximately 50%. However, while the genetic evidence is strong, the latter data suggests that an interplay with environmental factors also plays a key role in influencing T1D outcome.

Beta-cell replacement and regeneration strategies hold great promise for the treatment of Type I Diabetes (T1D) patients. Exogenous replacement of functional beta cells by islet transplantation has an established proof of concept, however is hampered by the lack of sufficient human donor pancreatic organs and the rapid functional decay of islets following the isolation procedure (Negi, 2011). Alternatively, regeneration approaches include activation of endogenous beta-cell proliferation and reprogramming of other cell types into beta cells (Dor 2004; Brennand 2009; Bonner-Weir 2000). These approaches can be limited by the poor function of derived endocrine cells, which often fail to maintain a differentiated phenotype.

The rapid functional decay of islet function is a consequence of the isolation procedure. Several features inherent in the islet isolation method contribute to the functional decline. As an example, unfavorable anoxic conditions occur as a result of ischemia between the time of the donor's death and the resection of the pancreas. In addition, mechanical stresses due to agitation and pipetting contribute to tissue damage. Furthermore, the enzymatic digestion steps required to enrich the desired endocrine cells from the surrounding exocrine tissue has deleterious effects on islet function. Hallmarks of this functional decline include increased apoptosis, a loss of insulin expression, and increased expression of inflammatory cytokines. The latter effect can further impede the survival of the islet transplant by stimulating local immune responses in the recipient. Clearly, a need exists for improved culture methods and islet cell compositions for use in islet transplantation. Specifically, improving isolation methods and culture conditions to increase both the yield and function of pancreatic islets will expand the utility of beta-cell replacement strategies used in the treatment of T1D, chronic pancreatitis, and potentially T2D.

### SUMMARY OF THE INVENTION

A method for culturing isolated pancreatic islets of Langerhans and islet cells derived from alternative biological sources is provided. The instant methods generate sufficient cells numbers for transplant, autotransplant, and investigative purposes. Islet cells can be obtained from cadaver donor pancreas, or obtained from stem cells induced to differentiate into beta islet cells. Also provided are improved islet cell populations generated using the methods of the invention. The islets/islet cells are treated with the agents disclosed herein during initial isolation and/or during culture. Such treatments include without limitation, incubation with pharmacological agents (e.g., estrogen signaling modulating agents, aromatase inhibitors, and antioxidants) which impact signaling pathways associated with robust islet cell proliferation and maintenance. Alternatively, genetic approaches introducing nucleic acids which encode molecules modulating such pathways (e.g., dominant negative estrogen receptor mutants) can be used. In preferred embodiments, agents or nucleic acids which modulate the estrogen signaling and activity of estrogen receptor alpha (ESR1) are employed. In a particularly preferred embodiment, the agent is raloxifene.

### BRIEF DESCRIPTION OF THE DRAWING

FIG. 1: Inhibition of estrogen receptor signaling prevents dedifferentiation in cultured islets. Islets were either untreated or treated with an estrogen receptor selective estrogen receptor modulator (SERM; 10 nM Raloxifene). RNA-Seq analysis of cultured islets was performed following 4 days in culture. Raloxifene treatment was for one day on the final day of culture. Expression levels of individual genes were calculated by the method of RPKM (reads per kilobase per million mapped reads). Data is represented as the log of the ratio (Islets-Raloxifene: Islets-no treatment) where positive values indicate an increase in gene expression with Raloxifene treatment. Interestingly, by inhibiting estrogen receptor activity with this antagonist, the increase in expression of inflammatory genes and genes associated with a dedifferentiation profile was prevented. (IL-8, CXXL6, SERPINB2, OLFM4). Raloxifene was without effect on housekeeping genes (GAPDH, ACTB, B2M) and in fact, increased expression of genes associated with a differentiated phenotype (SYT8, INS).

FIG. 2: Ectopic expression of ESR1 in an ESR1-deficient beta-cell model replicates the deleterious effects of activating estrogen signaling initially observed in isolated human islets. The immortalized beta-cell line INS1E does not express ESR1 and is not responsive to estrogenic compounds. Strikingly, ectopic expression of ESR1 mimics the toxic effects observed in isolated islets, namely decreased insulin mRNA and protein levels.

FIG. 3. Splice variant specific effects of estrogenic agents. In the previously described ESR1 deficient beta-cell model, three ESR1 isoforms (36, 46, and 66 kDa) were ectopically expressed to reveal differential potencies to estrogenic compounds. Estrogen receptor transactivation is measured with an ERE-luciferase reporter (Tzukerman, 1994) and apoptotic activity is assessed with a caspase activity assay (Préaudat, 2002). In cells lacking expression of any ESR1 isoform (control group), ERE-transcription (Panel A) was below detectable levels and caspase activity (Panel B) is low. Similar to the control, expression of ESR1-36 does not increase transcription nor caspase activities, which is consistent with lacking both transactivation domains. Expression of ESR1-46 increases both transcription and caspase activities compared to the control group. These increased activities are further potentiated with the agonist estradiol (E2) and near completely inhibited by the ligand antagonist MPP (1,3-Bis(4-hydroxyphenyl)-4-methyl-5-[4-(2-piperidinylethoxy)phenol]-1H-pyrazole dihydrochloride). In contrast to ESR1-46, E2 weakly affects ESR1-66 stimulation of transcription and caspase activities. MPP was less effective at suppressing transcription (65%) and caspase (40%) activities. This drawing demonstrates that antagonists of estrogen signaling reduce apoptosis and ESR1-isoforms can have unique sensitivities to such compounds.

### DETAILED DESCRIPTION OF THE INVENTION

Several strategies to improve islet yield and function have been evaluated to date (see Dauod 2010). Of these, a wide range of culture media supplements have been tested including growth factors (HGF, VEGF, IGF-I), antioxidants (Selenium, Ascorbic Acid, Vitamin D), minerals (Magnesium, Zinc) and fuel sources (Glucose, Pyruvate, Glutamate). One particular approach that has promise is the use of natural and synthetic hormones that have demonstrated protective effects against diabetes. Estrogen treatment improves insulin sensitivity in a number of diabetic animal models and as suggested by several groups can improve beta-cell function. In one study, streptortocin (STZ)-induced insulin deficiency in mice was exacerbated when estradiol production was suppressed. Beta-cell apoptosis was increased under these conditions, which was reversed with estradiol treatment (Le May, 2006). In the Zucker diabetic fatty rat (ZDF) model, estradiol treatment of the rats reduced islet lipid synthesis, increased beta-cell mass, and increased islet insulin content (Tiano, 2011). In isolated mouse islets, estradiol treatment increased insulin content and glucose-stimulated insulin secretion (Alonso-Magdalena, 2008). These beneficial effects of estradiol were absent in islets isolated from mice lacking ESR1 expression (Alonso-Magdalena, 2008). Experiments in isolated human islets demonstrated that islet death induced by proinflammatory cytokine treatment was suppressed by estradiol administration (Contreras, 2002). These results were corroborated in MIN6 cells and human islets where estradiol treatment protected against hydrogen peroxide-induced apoptosis (Liu, 2009). This study also concluded that the protective effects of estradiol were independent of the genomic activity of the estrogen receptor (Liu, 2009). Altogether, the dogma in the islet biology field is estrogenic compounds protect pancreatic beta-cells from a variety of stresses including lipid accumulation, cytokine insult, and oxidative stress. These studies also demonstrate that activation of estrogen signaling pathways can suppress apoptosis and this action is independent of estrogen receptor's role in transcriptional activation. The prevailing literature indicates that treatments which activate estrogen signaling provide good candidates to improve yield and function of human islets (Lui, 2013).

To evaluate if estrogenic compounds could attenuate the toxicity observed with the islet isolation process, human islet cultures were supplemented with estrogenic agonists and antagonists, It has been surprisingly discovered that inhibitors of estrogen signaling can prevent the increased inflammatory gene expression profile observed on freshly isolated human pancreatic islets. This novel observation is in direct opposition of the expectation from rodent models. It also has been surprisingly discovered that administration of inhibitors of estrogen signaling can improve the function of pancreatic beta-cells, including increasing insulin expression. Moreover, different isoforms of estrogen receptors can influence the potency of inhibitors of estrogen signaling on these effects. For example in prior in vivo mouse experiments, estrogen has a confounding effect on insulin sensitivity. By altering liver metabolism, estrogen can reduce hyperglycemia. Notably, hyperglycemic conditions are particularly deleterious to the islet. Thus the protective effects of estrogens on islets is likely mediated through reduced systemic hyperglycemia rather than via direct action on estrogen receptors in the beta-cell as has been previously proposed. A similar experimental design issue may have confounded the conclusions reached using isolated human islets in the in vivo (again hyperglycemic) mouse model described in Lui et al. 2013. In two published instances, estrogen treatment was shown to improve human islet function using similar oscillating protocols to this study. However the caveat from these studies is that to observe a protective effect of estrogen, it was necessary to dramatically induce cell death either by pro-inflammatory cytokines or a harsh hydrogen peroxide treatment. Such treatments would not be applicable for the preparation of islets for transplants.

As mentioned above, pancreatic islet isolation and culture induce a deleterious inflammatory response and dedifferentiation gene expression profiles (Negi, 2011). As a consequence, insulin content is reduced and secretagogue stimulated insulin release is impaired. In accordance with the present invention methods are provided for improving stability and endocrine functions of isolated pancreatic beta islet cells for the treatment of diabetes. Cells so treated can be used to advantage in transplantation protocols for the treatment and management of diabetes mellitus.

The following definitions are provided to facilitate an understanding of the present invention.

The phrase “Type 1 diabetes (T1D)” refers to a chronic (lifelong), autoimmune disease that occurs when the pancreas produces too little insulin to regulate blood sugar levels appropriately. T1D, often called juvenile or insulin-dependent diabetes results from altered metabolism of carbohydrates (including sugars such as glucose), proteins, and fats. In type 1 diabetes, the beta cells of the pancreas produce little or no insulin, the hormone that allows glucose to enter body cells. Once glucose enters a cell, it is used as fuel. Without adequate insulin, glucose builds up in the bloodstream instead of going into the cells. The body is unable to use this glucose for energy despite high levels in the bloodstream, leading to increased hunger. In addition, high levels of glucose in the blood causes the patient to urinate more, which in turn causes excessive thirst. Within 5 to 10 years after diagnosis, the insulin-producing beta cells of the pancreas are completely destroyed, and no more insulin is produced. The methods and compositions of the present invention may also be useful for the treatment of Type II diabetes.

Type 1 diabetes can occur at any age, but it usually starts in people younger than 30. Symptoms are usually severe and occur rapidly. The exact cause of type 1 diabetes is not known. Type 1 diabetes accounts for 3% of all new cases of diabetes each year. There is 1 new case per every 7,000 children per year. New cases are less common among adults older than 20.

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably.

Furthermore, a compound “selected from the group consisting of” refers to one or more of the compounds in the list that follows, including mixtures (i.e. combinations) of two or more of the compounds. The terms “compound and agent” are used interchangeably herein to refer to small molecules, pharmaceuticals, nucleic acid molecules (e.g., siRNA, shRNA, miRNA, antisense) or any substance which modulates a particular biochemical pathway or phenotypic process. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the functional and novel characteristics of the sequence.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10-6-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tm=81.5″C+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tm is 57″C. The Tm of a DNA duplex decreases by 1-1.5″C with every 1% decrease in homology. Thus, targets with greater than about 75% sequence identity would be observed using a hybridization temperature of 42″C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tm of the hybrid. Wash conditions should be as stringent as possible for the degree of identity of the probe for the target. In general, wash conditions are selected to be approximately 12-20° C. below the Tm of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “primer” as used herein refers to an oligonucleotide, either RNA or DNA, either single stranded or double stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non complementary nucleotide sequence may be attached to the 5′ end of an otherwise complementary primer. Alternatively, non complementary bases may be interspersed within the oligonucleotide primer sequence, provided that the primer sequence has sufficient complementarity with the sequence of the desired template strand to functionally provide a template primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like.

The term “autologous cells” as used herein refers to donor cells which are genetically compatible with the recipient.

“Multipotent” implies that a cell is capable, through its progeny, of giving rise to several different cell types found in the adult animal.

“Pluripotent” implies that a cell is capable, through its progeny, of giving rise to all the cell types which comprise the adult animal including the germ cells. Both embryonic stem and embryonic germ cells are pluripotent cells under this definition.

The term “cultured” as used herein in reference to cells can refer to one or more cells that are undergoing cell division or not undergoing cell division in an in vitro environment. An in vitro environment can be any medium known in the art that is suitable for maintaining cells in vitro, such as suitable liquid media or agar, for example. Specific examples of suitable in vitro environments for cell cultures are described in Culture of Animal Cells: a manual of basic techniques (3.sup.rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. Morgan John Wiley and Sons, Ltd.

The term “cell line” as used herein can refer to cultured cells that can be passaged at least one time without terminating. The invention relates to cell lines that can be passaged indefinitely. Cell passaging is defined hereafter.

The term “suspension” as used herein can refer to cell culture conditions in which cells are not attached to a solid support. Cells proliferating in suspension can be stirred while proliferating using apparatus well known to those skilled in the art.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells of the invention can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel which has been supplemented with medium suitable for further cell proliferation.

The term “proliferation” as used herein in reference to cells can refer to a group of cells that can increase in number over a period of time.

The term “embryonic stem cell” as used herein can refer to pluripotent cells isolated from an embryo that are maintained in in vitro cell culture. Such cells are rapidly dividing cultured cells isolated from cultured embryos which retain in culture the ability to give rise, in vivo, to all the cell types which comprise the adult animal, including the germ cells. Embryonic stem cells may be cultured with or without feeder cells. Embryonic stem cells can be established from embryonic cells isolated from embryos at any stage of development, including blastocyst stage embryos and pre-blastocyst stage embryos. Embryonic stem cells may have a rounded cell morphology and may grow in rounded cell clumps on feeder layers. Embryonic stem cells are well known to a person of ordinary skill in the art. See, e.g., WO 97/37009, entitled “Cultured Inner Cell Mass Cell-Lines Derived from Ungulate Embryos,” Stice and Golueke, published Oct. 9, 1997, and Yang & Anderson, 1992, Theriogenology 38: 315-335. See, e.g., Piedrahita et al. (1998) Biol. Reprod. 58: 1321-1329; Wianny et al. (1997) Biol. Reprod. 57: 756-764; Moore & Piedrahita (1997) In Vitro Cell Biol. Anim. 33: 62-71; Moore, & Piedrahita, (1996) Mol. Reprod. Dev. 45: 139-144; Wheeler (1994) Reprod. Fert. Dev. 6: 563-568; Hochereau-de Reviers & Perreau, Reprod. Nutr. Dev. 33: 475-493; Strojek et al., (1990) Theriogenology 33: 901-903; Piedrahita et al., (1990) Theriogenology 34: 879-901; and Evans et al., (1990) Theriogenology 33: 125-129.

The term “differentiated cell” as used herein can refer to a precursor cell that has developed from an unspecialized phenotype to a specialized phenotype. For example, embryonic cells can differentiate into an epithelial cell lining the intestine. Materials and methods of the invention can reprogram differentiated cells into totipotent cells. Differentiated cells can be isolated from a fetus or a live born animal, for example.

The term “undifferentiated cell” as used herein can refer to a precursor cell that has an unspecialized phenotype and is capable of differentiating. An example of an undifferentiated cell is a stem cell.

The term “feeder cells” as used herein can refer to cells that are maintained in culture and are co-cultured with target cells. Target cells can be precursor cells, embryonic stem cells, embryonic germ cells, cultured cells, and totipotent cells, for example. Feeder cells can provide, for example, peptides, polypeptides, electrical signals, organic molecules (e.g., steroids), nucleic acid molecules, growth factors (e.g., bFGF), other factors (e.g., cytokines such as LIF and steel factor), and metabolic nutrients to target cells. Certain cells, such as embryonic germ cells, cultured cells, and totipotent cells may not require feeder cells for healthy growth. Feeder cells preferably grow in a mono-layer.

Feeder cells can be established from multiple cell types. Examples of these cell types are fetal cells, mouse cells, Buffalo rat liver cells, and oviductal cells. These examples are not meant to be limiting. Tissue samples can be broken down to establish a feeder cell line by methods well known in the art (e.g., by using a blender). Feeder cells may originate from the same or different animal species as precursor cells. Feeder cells can be established from ungulate fetal cells, mammalian fetal cells, and murine fetal cells. One or more cell types can be removed from a fetus (e.g., primordial germs cells, cells in the head region, and cells in the body cavity region) and a feeder layer can be established from those cells that have been removed or cells in the remaining dismembered fetus. When an entire fetus is utilized to establish fetal feeder cells, feeder cells (e.g., fibroblast cells) and precursor cells (e.g., primordial germ cells) can arise from the same source (e.g., one fetus).

The term “promoter element” describes a nucleotide sequence that is incorporated into a vector that, once inside an appropriate cell, can facilitate transcription factor and/or polymerase binding and subsequent transcription of portions of the vector DNA into mRNA. Host cell machinery then translates mRNA into a polypeptide.

Those skilled in the art will recognize that a nucleic acid vector can contain nucleic acid elements other than the promoter element and the gene encoding the nucleic acid molecule of interest. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Exemplary siRNAs which modulate estrogen signaling pathways are described in Helguero et al. Ocogene 24:6605 (2005). Briefly, pSUPER (Oligoengine, Seattle, Wash., USA) was used to express the siRNAs. The sequences were chosen with ‘siRNA Selection Program’ (Whitehead Institute for Biomedical Research, 2003). The custom-designed oligos were from DNA Technology A/S, Denmark. The sequence to block ERa (siERa) was 50-GCTCCTGTTTGCTCCTAAC-30 (homologous to by 1395-1417 of the mouse ERa cDNA) and to block ERb (siERb) was 50-GTGCCAGCGAG CAGGTGCA-30 (homologous to by 472-494 of the mouse ERb cDNA). The specificity of these oligos was controlled by inserting a point mutation in base 9 (T for G in mutsiERa and G for T in mutsiERb). Generation of the plasmids was carried out following the manufacturer's suggestions (oligoengine.com).

In the experiments described, HC11 cells were grown in complete medium until 50% confluence, and then transfection was performed with Fugene (Roche). A double transient transfection was carried out to control the blockade efficiency of each siRNA: 1 mg pCMXYFP-mERa (YFP-mERa) with 0.5 mg siERa or mutsiERa; alternatively the same amounts of pCMX-GFP-mERb ERa and ERb in cell proliferation and apoptosis (GFP-mERb) with siERb or mutsiERb. The cells were grown for 24 h, fixed in 10% buffered formalin and differences between YFP or GFP intensity were evaluated by observation of random fields under the same microscope settings. To control the specificity of siRNAs in the response to E2 in cell counting experiments, cotransfection of 0.5 mg of each pSUPER-siRNA plasmid together with 100 ng of pGL3-control vector coding the firefly luciferase gene (Promega) was performed. Cells were simultaneously transfected and treated with or without 10 nM E2. After 2 days, cells were counted and number of cells/ml was normalized to luciferase activity in each well. Two independent experiments were carried out, each comprising sextuplicates for each treatment. Following normalization, proliferation index was calculated comparing to the untreated control within the same experiment, which was arbitrarily set to 1.

An estrogen signaling modulator compound as used herein refers to any agent effective to modulate estrogen mediated cellular effects or cellular signaling pathways thereby maintaining the endocrine phenotype of beta islet cells. Other compounds include but are not limited to those set forth in Table I below.

TABLE I
DRUGS TARGETING ESTROGEN-ER PATHWAY AND DRUG
STATUS

Fulvestrant approved antagonist
Ormeloxifene approved antagonist
Raloxifene approved
Tamoxifen approved agonist
Toremifene approved agonist
Arzoxifene in clinical trials antagonist
Bazedoxifene approved in Europe antagonist
Pipendoxifene in clinical trials antagonist
Lasofoxifene approved in Europe
HMR-3339 in clinical trials undefined
Ospemifene in clinical trials agonist
Acolbifene (EM-652) in clinical trials antagonist
EM-800 clinical development antagonist
Droloxifene clinical development agonist
Idoxifene clinical development agonist
Levormeloxifene clinical development agonist

Additional anti-estrogen compounds include, without limitation, ICI 182780 and ICI 164384.

In another approach for inhibiting estrogen action, dominant negative estrogen receptor mutant molecules can be introduced into pancreatic beta islet cells via transfection. Such mutants are described in Ince et al. (Endocrinology 136:3194-9 (1995)). Adenoviral vectors suitable for use in this approach are described in Lazennec et al. (Mol. Endocrinology 13:969-80 (1999).

Several different classes of agents can also be employed with the SERMs of the invention for improving beta islet cell function. These include without limitation, superoxide dismutase mimics, agents which effect redox modulation, and aromatase inhibitors. Such agents should act synergistically with the SERMS to improve and stabilize islet cells.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

As used herein, “treating” a condition or patient refers to taking steps to obtain beneficial or desired results, including clinical results. For purposes of this invention, beneficial or desired clinical results include, but are not limited to, alleviation or amelioration of one or more symptoms of diabetes, diminishment of extent of disease, delay or slowing of disease progression, amelioration, palliation or stabilization of the disease state, and other beneficial results described below.

As used herein, “reduction” of a symptom or symptoms (and grammatical equivalents of this phrase) means decreasing of the severity or frequency of the symptom(s), or elimination of the symptom(s).

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “manifestation” of a disease refers to a symptom, sign, anatomical state (e.g., lack of islet cells), physiological state (e.g., glucose level), or report (e.g., triglyceride level) characteristic of a subject with the disease.

As used herein, a “therapeutically effective amount” of a drug or agent is an amount of a drug or agent that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a prophylactically effective amount may be administered in one or more administrations.

“Sample” or “patient sample” or “biological sample” generally refers to a sample if islet or islet cells for use in the methods of the present invention.

### Methods for Isolation of Beta Islet Cells for Use in the Present Invention

Several protocols are provided hereinbelow for the isolation and purification of islet cells.

In one approach, the pancreas can be distended intraductally with a cold enzyme (Liberase HI; Roche Applied Science, Indianapolis, Ind.). After delivery of enzyme solution, the pancreas can be transferred to a special container designed to facilitate tissue digestion. The islets are then separated by gentle enzymatic and mechanical dissociation. At intervals during the digestion, samples are taken to monitor dissociation of the pancreas. When an adequate number of acinar-free islets are found, the enzymatic process is stopped by cooling and dilution and the crude cellular fraction concentrated by centrifugation. The cooled crude extract containing islet cell clusters can then be purified with the use of Ficoll gradients in a refrigerated cell processor (COBE 2991; COBE Laboratories, Inc., Lakewood, Colo.). See Transplantation 82: 1286-1290 (2006).

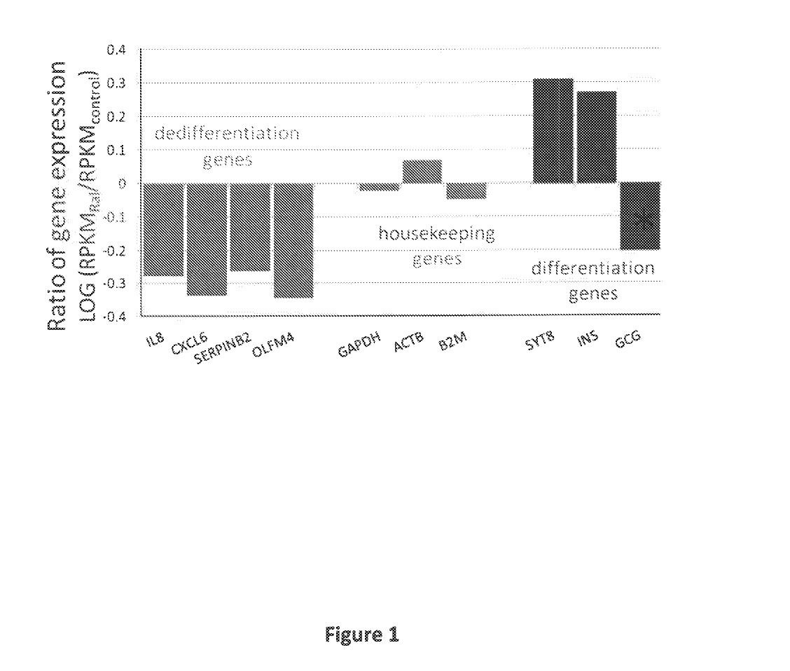

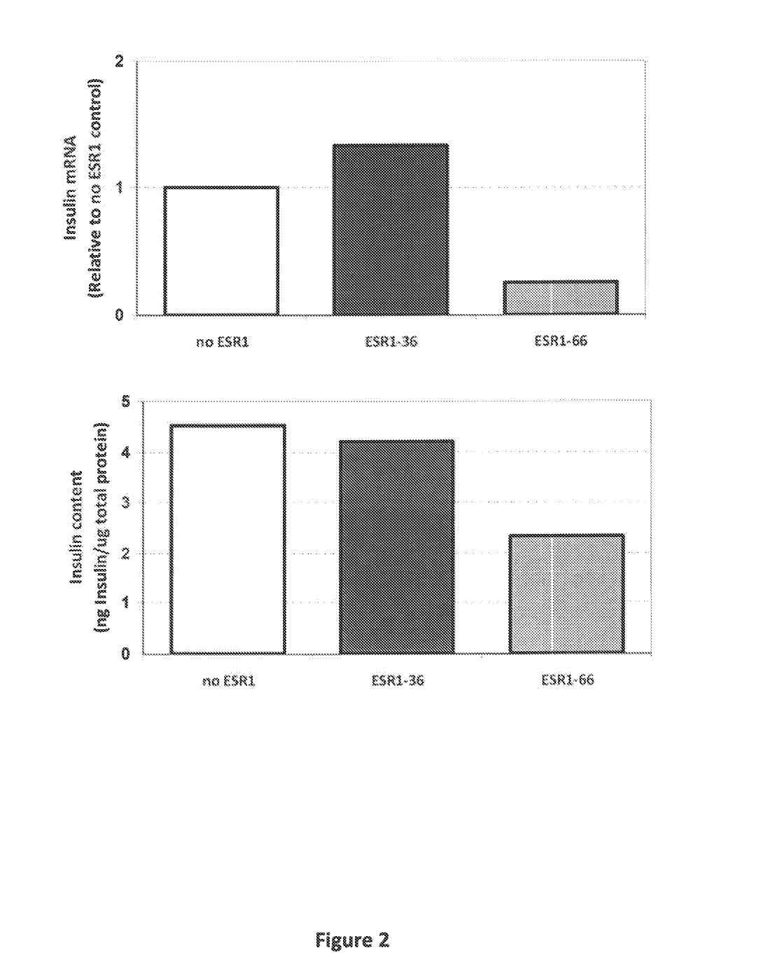

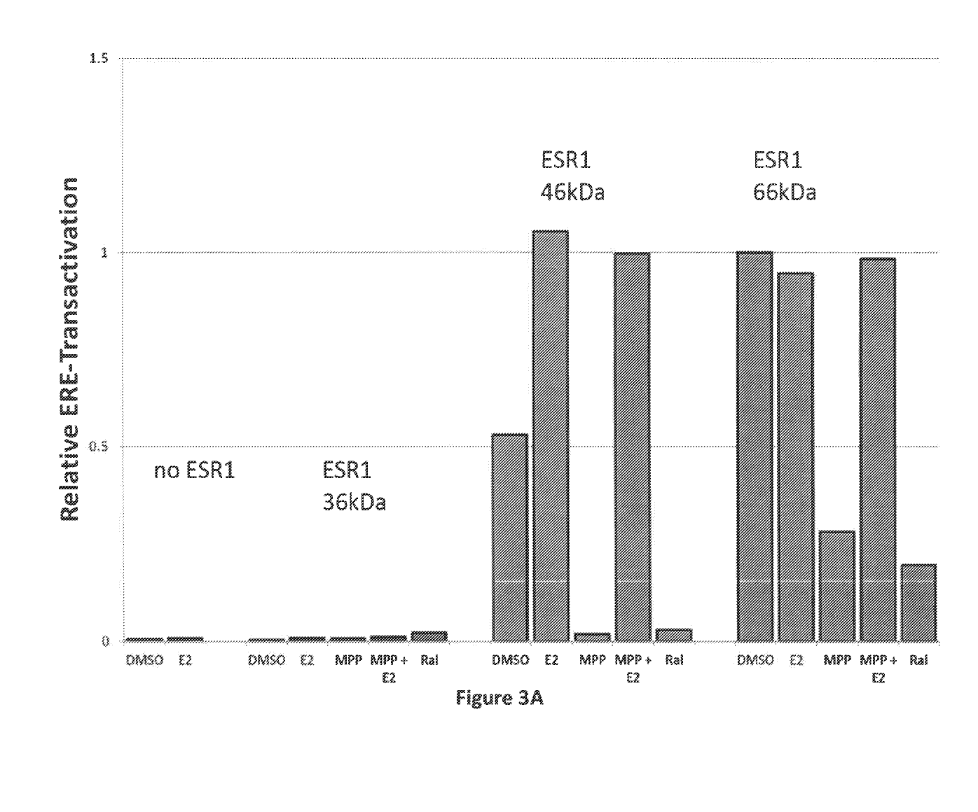

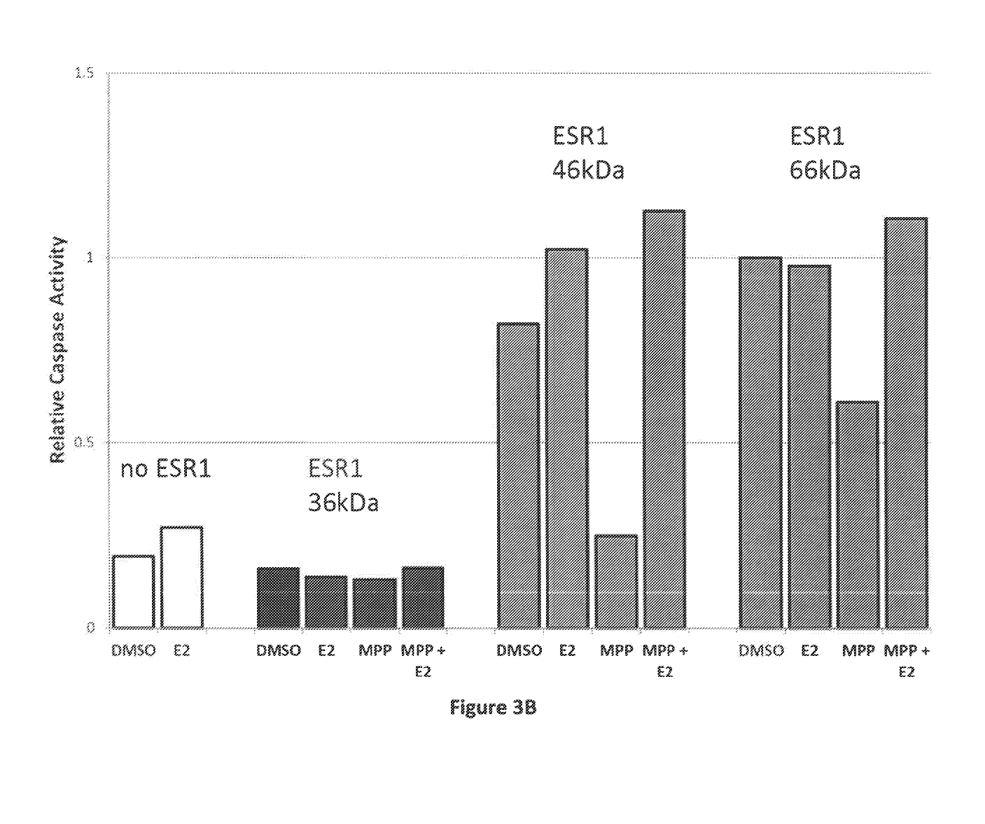

Following isolation, the islet cell clusters are cultured in Connaught Medical Research Laboratory 1066 medium supplemented with 10 nM Raloxifene. These are the conditions described in the legend to FIG. 1.

### Table 2.

[symbol]

An inflammatory gene expression profile is evoked following human islet isolation and culture and suppressed by Raloxifene treatment. Gene expression was assessed in isolated cultured human islets on multiple days following the isolation procedure. Islets were cultured in standard CMRL media with or without the addition of Raloxifene and gene expression profiles were assessed by RNA-Seq. In table 2A, the progressive increase of NFkB-target gene expression is observed over consecutive days in culture. Of note, significant increases in canonical NFkB targets (CCL2, CCL20, IL1B) as well as NFkB coordinately regulated gene clusters, such as the innate immunity gene cluster (APOL1-APOL6) were observed. In table 2B, Raloxifene treatment (10 nM) suppressed gene expression of the innate immunity gene cluster (APOL1-APOL6) which is typically observed to increase with the isolation and culture of human islets. Gene expression was assessed with RNA-Seq and quantification of gene transcripts is represented as RPKM (reads per kilobase mapped). Note, three genes (APOL1, APOL2, and APOL6) in the innate immunity gene cluster were each reduced by Raloxifene treatment.

TABLE 2A
Human islet
isolation and triggers an
inflammatory gene
expression profile

| | | |
|---|---|---|
| Gene | Fold | p value |

NFkB targets

| | | |
|---|---|---|
| CCL2 | 10.9 | 0.0002 |
| CCL20 | 8.8 | 0.0044 |
| IL1B | 3.9 | 0.0070 |

APOL gene cluster

| | | |
|---|---|---|
| APOL1 | 7.4 | 0.0044 |
| APOL2 | 3.4 | 0.0154 |
| APOL3 | 1.2 | 0.8727 |
| APOL4 | LD | — |
| APOL5 | ND | — |
| APOL6 | 7.5 | 0.0002 |

LD = low detection;
ND = not detected

TABLE 2B
Raloxifene treatment of cultured
isolated human islets suppresses the gene
expression of the innate immunity gene
cluster APOL1-APOL6.

| | RPKM | RPKM | % decrease in |
|---|---|---|---|
| Gene | (control) | (Ral) | gene expression |
| APOL1 | 2.1 | 1.8 | 15% |
| APOL2 | 13.6 | 4.3 | 68% |
| APOL6 | 1.5 | 0.9 | 41% |

The Edmonton Protocol refers to immunosuppression therapy utilized to enable human islet transplantation. See Shapiro et al. (2000). These investigators disclose treatment of seven consecutive patients with type 1 diabetes and a history of severe hypoglycemia and metabolic instability with islet transplantation in conjunction with a glucocorticoid-free immune-suppressive regimen consisting of sirolimus, tacrolimus, and daclizumab. Islets were isolated by ductal perfusion with cold, purified collagenase, digested and purified in xenoprotein-free medium, and transplanted immediately by means of a percutaneous transhepatic portal embolization. These investigators further report that all seven patients quickly attained sustained insulin independence after transplantation of a mean (+/−SD) islet mass of 11,547+/−1604 islet equivalents per kilogram of body weight (median follow-up, 11.9 months; range, 4.4 to 14.9). All recipients required islets from two donor pancreases, and one required a third transplant from two donors to achieve sustained insulin independence. The mean glycosylated hemoglobin values were normal after transplantation in all recipients. The mean amplitude of glycemic excursions (a measure of fluctuations in blood glucose concentrations) was significantly decreased after the attainment of insulin independence (from 198+/−32 mg per deciliter [11.1+/−1.8 mmol per liter] before transplantation to 119+/−37 mg per deciliter [6.7+/−2.1 mmol per liter] after the first transplantation and 51+/−30 mg per deciliter [2.8+/−1.7 mmol per liter] after the attainment of insulin independence; P<0.001). There were no further episodes of hypoglycemic coma. Complications were minor, and there were no significant increases in lipid concentrations during follow-up. However, while the Edmonton protocol can successfully restore long-term endogenous insulin production and glycemic stability in subjects with type 1 diabetes mellitus and unstable control, but insulin independence is usually not sustainable.

Another approach of improving beta islet cell function that can be used in conjunction with the estrogen modulating agents disclosed herein is described in Piganelli et al. (2002). These investigators describe a metalloporphyrin-based antioxidant which can prevent or delay the onset of autoimmune diabetes. Because islet beta-cells have a reduced capacity to scavenge free radicals, they are very sensitive to ROS action. Metalloporphyrin-based superoxide dismutase (SOD) mimics scavenge ROS and protect cells from oxidative stress and apoptosis. To investigate the effect of SOD mimics and the role of oxidative stress in the development of autoimmune diabetes in vivo, Pignalli et al. used a diabetogenic T-cell clone, BDC-2.5, to induce rapid onset of diabetes in young nonobese diabetic-severe combined immunodeficient mice (NOD.scid). Disease was significantly delayed or prevented altogether by treatment of recipient mice with an SOD mimic, AEOL-10113, before transfer of the BDC-2.5 clone.

Several laboratories have disclosed methods of generating pancreatic endoderm from stem cells. Accordingly, beta islet cells prepared from stem cells are also encompassed by the present invention. In one approach, described in Moshtagh et al., human adipose-derived mesenchymal stem cell were successfully differentiated into insulin-producing cells Mesenchymal stem cells (MSCs) are pluripotent stromal cells with the ability to proliferate and differentiate into a variety of cell types including endocrine cells of the pancreas. These investigators studied the in vitro differentiation of human adipose-derived tissue stem cells into IPCs which could provide an abundant source of cells for the purpose of diabetic cell therapy with the added benefit of avoiding immunological rejection. Adipose-derived MSCs were obtained from liposuction aspirates and induced to differentiate into insulin-secreting cells under a three-stage protocol based on a combination of low-glucose DMEM medium, β-mercaptoethanol, and nicotinamide for pre-induction and high-glucose DMEM, β-mercaptoethanol, nicotinamide, and exendin-4 for induction stages of differentiation. Differentiation was evaluated by the analysis of morphology, dithizone staining, RT-PCR, and immunocytochemistry. Morphological changes including typical islet-like cell clusters were observed by phase-contrast microscope at the end of differentiation protocol. Based on dithizone staining, differentiated cells were positive and undifferentiated cells were not stained. Furthermore, RT-PCR results confirmed the expression of insulin, PDX1, Ngn3, PAX4, and GLUT2 in differentiated cells. Moreover, insulin production by the IPCs was confirmed by immunocytochemistry analysis. The authors concluded that adipose-derived MSCs could differentiate into insulin-producing cells in vitro. Such cells could be further stabilized using the methods disclosed herein.

In another approach using embryonic stem cells in a murine system, ES cells were transfected by electroporation with a plasmid expressing β-gal under the control of the human insulin regulatory region and expressing the hygromycin resistance gene under the control of the pGK promoter. Transfected clones were selected by growth in the presence of hygromycin (200 μg/ml; Calbiochem-Novabiochem). Transfected ES cells were maintained in the undifferentiated state by culturing in high glucose Dulbecco's modified Eagle's medium (DMEM) containing 10% fetal bovine serum (FBS), 1% nonessential amino acids, 0.1 mM 2-mercaptoethanol, 1 mM sodium pyruvate, 100 IU/ml penicillin, and 0.1 mg/ml streptomycin. The medium was supplemented to a final concentration of 100 U/ml with conditioned medium containing recombinant LIF.

To induce differentiation to an insulin-secreting cell line, 2×10⁶ hygromycin-resistant ES cells were plated onto a 100-mm bacterial Petri dish and cultured in DMEM lacking supplemental LIF. After 8-10 days in suspension culture, the resulting EBs were plated onto plastic 100-mm cell culture dishes and allowed to attach for 5-8 days. For ES Ins/β-gal selection, the differentiated cultures were grown in the same medium in the presence of 200 μg/ml G418. For final differentiation and maturation, the resulting clones were trypsinized and plated on a 100-mm bacterial Petri dish and grown for 14 days in DMEM supplemented with 200 μg/ml G418 and 10 mM nicotinamide (Sigma), a form of Vitamin B3 that may preserve and improve beta cell function. Finally, the resulting clusters were cultured for 5 days in RPMI 1640 media supplemented with 10% FBS, 10 mM nicotinamide, 200 μg/ml G418, 100 IU/ml penicillin, 0.1 mg/ml streptomycin, and low glucose (5.6 mM). These protocols can be adapted to human cells which would then be suitable for implantation into subjects. The ES-derived insulin-secreting cells produced from this method produced a similar profile of insulin production in response to increasing levels of glucose to that observed in mouse pancreatic islets. Significantly, implantation of the ES-derived insulin-secreting cells led to the correction of the hyperglycemia within the diabetic mouse, minimized the weight loss experienced by the mice injected with STZ, and lowered glucose levels after meal challenges and glucose challenges better than untreated diabetic mice and similar to control nondiabetic mice.

A Scalable System for Production of Functional Pancreatic Progenitors from Human Embryonic Stem Cells is described by Schulz et al. in PLoS ONE 7(5): e37004. doi:10.1371/journal.pone.0037004 (2012). In this approach, undifferentiated hESC Culture (CyT49 hESC line, NIH registration number: 0041) was used in these studies. Xeno-free growth media (XF HA) consisted of DMEM/F12 containing GlutaMAX (Life Technologies, cat#10565) supplemented with 10% (v/v) of Xeno-free KnockOut Serum Replacement (Life Technologies, cat#12618-001), 1% (v/v) non-essential amino acids (Life Technologies, cat#11140-050), 0.1 mM 2-mercaptoethanol (Life Technologies, cat#21985-023), 1% (v/v) penicillin/streptomycin (Life Technologies, cat#15070-063), 10 ng/mL heregulin-1β (Peprotech, cat#100-03) and 10 ng/mL activin A (R & D Systems, cat#338-AC). Upon thaw, or at regular passaging, dissociated hESC were plated at 50,000 or 33,000 cells/cm² for three and four day growth cycles, respectively. On the day of plating only, cell attachment was facilitated by including 10% (v/v) of non-heat inactivated human AB serum (Valley Biomedical, cat#HP1022). A standardized plating volume of 0.2 mL/cm2 was used for different tissue culture plates, T-flasks and cell factories. The volume of growth media used was increased for each additional day of feeding, and did not include human serum. On the day of passaging, cultures were fed with fresh growth media and cultured 4-8 hrs before dissociation. Cultures were washed with PBS (Life Technologies, cat#10010-031) and dissociated for 6 mins at 37° C. using pre-warmed Accutase (Innovative Cell Technologies, cat# AT104). Dissociated cells were gently collected using a volume of cold hESC media (without heregulin or activin), counted using a ViCell automated cell counter (BD Biosciences), or a hemocytometer, centrifuged for 5 mins at 2006 g and the pellet resuspended in fresh growth media at 1−10×10⁶ cells/mL for subsequent plating. Where indicated, the StemPro hESC SFM medium (Life Technologies, cat#A1000701) supplemented with 10 ng/mL heregulin-1β, 10 ng/mL activin A, 10 ng/mL FGF2 and 200 ng/mL LR3-IGF1 (StemPro), or the same media without FGF2 (SP HAI) were used for hESC culture or suspension culture. Cell culture Cryopreservation and Banking of hESC Small and large-scale single-cell banks were cryopreserved using the same approach Adherent cell cultures were harvested according to the above passaging protocol, pooled and counted. Cell pellets were resuspended in prewarmed 50% hESC culture medium (without growth factors)/50% human serum. An equal volume of 80% hESC culture medium (without growth factors)/20% DMSO was added drop-wise, with swirling. 1 mL of cells was distributed to 1.8 mL Nunc cryovials for freezing at 280 uC in Nalgene Mr Frosty containers for 24 hrs, before transferring to liquid N2. cGMP culture and banking were performed by Viacyte employees at a certified 3rd party contract research organization.

### Aggregate Formation and Differentiation

Scaled pancreatic differentiation runs in suspension typically utilized 18-20 6-well trays and were carried out by first generating hESC aggregates. On the fourth day after passage adherent hESC cultures were fed with fresh XF HA media and cultured 4-8 hrs before dissociation. Cultures were harvested according to the passaging protocol and resuspended in approximately 10 mL cold XF HA media per 150 cm2 culture surface area. Cells were counted using a ViCell automated cell counter, then seeded into ultra-low adhesion 6-well trays (Greiner BioOne, cat#657185) at 1−6×10⁶ cells/mL in XF HA media and 5.5 mL per well. The 6-well trays were incubated at 37° C. on orbital rotators set at 95 rpm (Innova2000, New Brunswick Scientific). During overnight culture, single cells aggregated to form spherical clusters, approximately 100-200 mm in diameter and roughly 5,000 aggregates per well.

To initiate differentiation, aggregates were pooled into conical tube(s) and allowed to settle by gravity, or in some experiments by centrifugation at 500 rpm for 90 seconds, followed by a wash using media without growth factors. The aggregates were resettled, then resuspended in day 1 media and evenly redistributed to the same number of wells using new 6-well trays in 5.5 mL total volume per well. After 20-24 hrs of differentiation some cell death and a wispy mass, or clump, of DNA was visible in each well. On this day, clumps were disrupted by triturating with a P200 pipettor and returned to the rotator for 30 seconds to allow aggregates to collect in the center of the well. Approximately 4.5 mL of media was aspirated from each well and wells were fed with 4.5 mL of day 2 media. Aspiration of 4.5 mL of media followed by feeding with 4.5 mL of the subsequent day's media was performed daily thereafter. In some experiments, at the end of Stage-2 the aggregates were pooled and redistributed to half the original number of wells in 5.5 mL of day 5 media. Aggregates were sampled at the indicated time points for histological, immunofluorescence, and gene expression analyses

The following example is provided to illustrate certain embodiments of the invention. It is not intended to limit the invention in any way.

### EXAMPLE

With the goal to identify novel cellular signaling pathways that affect pancreatic beta-cell growth-differentiation-survival axes, a compound screen of approved FDA compounds was performed on pancreatic beta-cells. Lead compounds included a FDA approved selective estrogen receptor modulator, Raloxifene. Follow-up experimentation included gene expression analysis in human islets with and without the addition of Raloxifene in the culture media.

As shown in FIG. 1, striking changes in gene expression profiles were observed which included decreased expression of genes associated with inflammatory responses and increased expression of genes associated with differentiated endocrine cells. Thus, treatment of isolated islet cultures with Raloxifene, and like compounds, preserved the endocrine phenotype.

Improving the isolation and culture procedures of islets suitable for transplantation has significant therapeutic consequences. It is well recognized that the utility of human islets for transplantation decreases rapidly following isolation and culture. This is in part due to decreasing viability and increased dedifferentiation of the endocrine cells. The decreased functionality includes diminished stimulation of insulin release.

The methods of culture provided herein extend the critical time between isolation and transplant by preserving the endocrine phenotype.

Following transplantation, a significant proportion of islets are lost within hours likely due to an acute inflammatory response. We believe a proportion of the immune response is due to increased inflammatory gene expression induced by isolation and culture. The present method inhibits this undesirable increase in inflammatory gene expression thus mitigating acute inflammatory islet injury.

Regenerative approaches to derive endocrine cells from various cell sources have the potential to greatly improve the supply of pancreatic beta-cells for the treatment of diabetes. The critical barrier preventing this approach is the typically low level of differentiation of cells produced. The method described has a demonstrated impact on gene expression profiles of genes associated with a differentiated cellular phenotype.

### REFERENCES

Alonso-Magdalena P, Ropero A B, Carrera M P, Cederroth C R, Baquié M, Gauthier B R, Nef S, Stefani E, Nadal A. (2008) Pancreatic insulin content regulation by the estrogen receptor ER alpha. PLoS One. 3(4):e2069.

Bonner-Weir S, Taneja M, Weir G C, Tatarkiewicz K, Song K H, Sharma A, O'Neil J J. (2000) In vitro cultivation of human islets from expanded ductal tissue. PNAS 97(14):7999-8004.

Bottino, R., A. N. Balamurugan, H. Tse, C. Thirunavukkarasu, X. Ge, J. Profozich, M. Milton, A. Ziegenfuss, M. Trucco and J. D. Piganelli (2004). Response of human islets to isolation stress and the effect of antioxidant treatment. Diabetes 53(10): 2559-68.

Brennand K, Melton D. (2009) Slow and steady is the key to beta-cell replication. J Cell Mol Med. 13(3):472-87.

17beta-Estradiol protects isolated human pancreatic islets against proinflammatory cytokine-induced cell death: molecular mechanisms and islet functionality. (2002) Contreras J L, Smyth C A, Bilbao G, Young C J, Thompson J A, Eckhoff D E. Transplantation. 74(9):1252-9.

Dor Y, Brown J, Martinez O I, Melton D A. (2004) Adult pancreatic beta-cells are formed by self-duplication rather than stem-cell differentiation. Nature. 2004 429(6987):41-6.

Daoud J, Rosenberg L, Tabrizian M. (2010) Pancreatic islet culture and preservation strategies: advances, challenges, and future outlook. Cell Transplant. 19(12):1523-35.

Le May C, Chu K, Hu M, Ortega C S, Simpson E R, Korach K S, Tsai M J, Mauvais-Jarvis F. (2006) Estrogens protect pancreatic beta-cells from apoptosis and prevent insulin-deficient diabetes mellitus in mice. Proc Natl Acad Sci USA. 103(24):9232-7.

Liu S, Mauvais-Jarvis F. (2009) Rapid, nongenomic estrogen actions protect pancreatic islet survival. Islets. 1(3):273-5.

Liu S, Kilic G, Meyers M S, Navarro G, Wang Y, Oberholzer J, Mauvais-Jarvis F. (2013) Oestrogens improve human pancreatic islet transplantation in a mouse model of insulin deficient diabetes. Diabetologia. 56(2):370-81.

Negi S, Jetha A, Aikin R, Hasilo C, Sladek R, Paraskevas S. Analysis of beta-cell gene expression reveals inflammatory signaling and evidence of dedifferentiation following human islet isolation and culture. PLoS One. 2012;7(1):e30415

Paraskevas, S.; Maysinger, D.; Wang, R. N.; Duguid, W. P.; Rosenberg, L. (2000) Cell loss in isolated human islets occurs by apoptosis. Pancreas 20(3):270-276

Piganelli, J. D., S. C. Flores, C. Cruz, J. Koepp, I. Batinic-Haberle, J. Crapo, B. Day, R. Kachadourian, R. Young, B. Bradley and K. Haskins (2002). A metalloporphyrin-based superoxide dismutase mimic inhibits adoptive transfer of autoimmune diabetes by a diabetogenic T-cell clone. Diabetes 51(2): 347-55.

Préaudat, M. et al. (2002) A homogeneous caspase-3 activity assay using HTRF technology. J. Biomol. Screen. 7,267-74.

Shapiro, A. M., J. R. Lakey, E. A. Ryan, G. S. Korbutt, E. Toth, G. L. Warnock, N. M. Kneteman and R. V. Rajotte (2000). Islet transplantation in seven patients with type 1 diabetes mellitus using a glucocorticoid-free immunosuppressive regimen. N Engl J Med 343(4): 230-8.

Shapiro, A. M., C. Ricordi, B. J. Hering, H. Auchincloss, R. Lindblad, R. P. Robertson, A. Secchi, M. D. Brendel, T. Berney, D. C. Brennan, E. Cagliero, R. Alejandro, E. A. Ryan, B. DiMercurio, P. Morel, K. S. Polonsky, J. A. Reems, R. G. Bretzel, F. Bertuzzi, T. Froud, R. Kandaswamy, D. E. Sutherland, G. Eisenbarth, M. Segal, J. Preiksaitis, G. S. Korbutt, F. B. Barton, L. Viviano, V. Seyfert-Margolis, J. Bluestone and J. R. Lakey (2006). International trial of the Edmonton protocol for islet transplantation. N Engl J Med 355(13): 1318-30.

Soria B, Roche E, Berná G, León-Quinto T, Reig J A, Martin F. (2000) Insulin-secreting cells derived from embryonic stem cells normalise glycaemia in streptozotocin-induced diabetic mice. Diabetes. 49:1-6.

Tiano J P, Delghingaro-Augusto V, Le May C, Liu S, Kaw M K, Khuder S S, Latour M G, Bhatt S A, Korach K S, Najjar S M, Prentki M, Mauvais-Jarvis F. (2011) Estrogen receptor activation reduces lipid synthesis in pancreatic islets and prevents β cell failure in rodent models of type 2 diabetes. J Clin Invest. 121(8):3331-42.

Tzukerman M T, Esty A, Santiso-Mere D, Danielian P, Parker M G, Stein R B, Pike J W, McDonnell D P. (1994) Human estrogen receptor transactivational capacity is determined by both cellular and promoter context and mediated by two functionally distinct intramolecular regions. Mol Endocrinol 8:21-30

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for in vitro culturing isolated pancreatic islets of Langerhans and/or endocrine cells isolated therefrom suitable for transplantation into human subjects, comprising:
providing viable pancreatic islets of Langerhans cells which maintain a differentiated phenotype and produce insulin,
culturing said cells in a basal medium supplemented with at least one first compound which modulates estrogen signaling and optionally at least one second compound effective to promote islet cell function and stability, said first compound being effective to prolong endocrine activity in said islet relative to untreated islet cells.

2. The method of claim 1, wherein the first compound is an anti-estrogen compound which modulates estrogen signaling via estrogen receptor alpha (ESR1) and is selected from the group consisting of ICI 182780 and ICI 164384.

3. The method of claim 1, wherein the first compound modulates estrogen signaling via selective activity against estrogen receptor alpha (ESR1) isoforms which are derived from mRNA splice variation.

4. The method of claim 1, wherein the first compound is 1,3-Bis(4-hydroxyphenyl)-4-methyl-5-[4-(2-piperidinylethoxy)phenol]-1H-pyrazole dihydrochloride.

5. The method of claim 1, comprising genotyping of the cells wherein said genotyping entails RNA analysis.

6. (canceled)

7. The method of claim 1, comprising protein analysis of the cells.

8. The method of claim 1, wherein maintaining a differentiated phenotype includes prevention of apoptosis and/or suppression of an inflammatory response.

9. (canceled)

10. The method of claim 1, wherein said first compound modulates steroid or estradiol biosynthesis and is selected from the group consisting of Femara®, and Aromasin®.

11. The method of claim 1, wherein said first compound is a selective estrogen receptor modulator selected from the group consisting of Nolvadex®, and Evista®.

12. The method claim 1 wherein activity of at least one of estrogen receptor beta (ESR2), and G-protein-coupled estrogen receptor 1 (GPER is modulated.

13-14. (canceled)

15. The method of claim 1 wherein the Langerhans islets are dispersed and obtained from a subject selected from the group consisting of human, pig, monkey, rat, and mouse.

16. The method of claim 1 wherein endocrine cells of the Langerhans islets are derived from stem cells.

17. The method of claim 1, wherein endocrine cells are obtained from cells selected from the group consisting of iPS, AFS, adipose cells and hepatocytes.

18. The method of claim 1 wherein the second compound is serum.

19. The method of claim 1 wherein the second compound is present and comprises one or more growth factors.

20. The method of claim 1 wherein estrogen signaling is modulated via overexpression and/or expression of dominant-negative transgenes.

21. The method of claim 20 wherein estrogen signaling is modulated via RNA interference.

22. The method of claim 1 wherein activity of estrogen signaling gene targets are modulated.

23. The method of claim 1 wherein steroid hormone levels of endocrine cells are modulated.

24. The method of claim 1 wherein modulation of estrogen signaling is initiated during the isolation procedure.

25. The method of claim 1, wherein said second compound is present and is selected from an antioxidant and, an aromatase inhibitor.

26. (canceled)

27. The method of claim 1, wherein said first compound is raloxifene.

28. The method of claim 1 wherein activity of at least one of estrogen-related receptor alpha (ESRRA), estrogen-related receptor beta (ESRRB), and estrogen-related receptor gamma (ESRRG) is modulated.

29-30. (canceled)

31. A stabilized islet cell population isolated using the method of claim 1, said population exhibiting endocrine activity and optionally secreting insulin.

32. (canceled)

33. A method for the treatment of diabetes comprising administration of an effective amount of the cell population of claim 31 to a subject in need thereof, said cell population being effective to increase insulin production in said subject.
